(19) United States
(12) Patent Application Publication
(10) Pub. No.: US 2025/0144423 A1
(43) Pub. Date: May 8, 2025

(54) METHODS AND APPARATUSES FOR REDUCING BLEEDING VIA COORDINATED TRIGEMINAL AND VAGAL NERVE STIMULATION

(71) Applicant: The Feinstein Institutes for Medical Research, Manhasset, NY (US)

(72) Inventor: Kirk R. MANOGUE, Manhasset, NY (US)

(21) Appl. No.: 19/002,650

(22) Filed: Dec. 26, 2024

Related U.S. Application Data
(63) Continuation of application No. 17/578,339, filed on Jan. 18, 2022, now Pat. No. 12,220,579.
Application No. 17/578,339 is a continuation of application No. 16/582,726, filed on Sep. 25, 2019, now Pat. No. 11,260,229.
(60) Provisional application No. 62/736,447, filed on Sep. 25, 2018.

(51) Int. Cl. A61N 1/36; A61N 1/04; A61N 1/05

(52) U.S. Cl. CPC A61N 1/36053; A61N 1/0456; A61N 1/0526; A61N 1/36017; A61N 1/36036; A61N 1/3606

(57) ABSTRACT

Disclosed are apparatuses and methods for reducing or limiting blood loss and reducing bleed time in a subject by combined vagus and trigeminal stimulation. The apparatuses and methods may activate (e.g., electrically) one or more branches of the trigeminal nerve and may concurrently (at overlapping or near-overlapping time) independently activate the vagus nerve. This activation may be invasive or non-invasive.

### CROSS REFERENCE TO RELATED APPLICATIONS

This patent application is a continuation of U.S. patent application Ser. No. 17/578,339, filed Jan. 18, 2022, now U.S. Patent Application Publication No. 2022/0212012, which is a continuation of U.S. patent application Ser. No. 16/582,726, filed Sep. 25, 2019, now U.S. Pat. No. 11,260,229, which claims priority to U.S. Provisional Patent Application No. 62/736,447, filed on Sep. 25, 2018, each of which are herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure is generally related to preventing and/or treating bleeding in a subject. More specifically, this disclosure is related to apparatuses (devices, systems, and methods) for preventing and/or treating bleeding and decreasing bleed time in a patient through stimulation of both the trigeminal and vagus nerves, such as through electrical and/or mechanical stimulation of the trigeminal and vagal nerve.

### BACKGROUND

Blood is an essential component of an animal's body to transport oxygen, nutrients and waste, protect the body with white blood cells and other immune system functions, and regulate body functions such as pH and hydration within critical ranges. Blood loss may lead to a variety of problems, including dysregulation or ultimately death. Blood loss can occur due to a various causes. For example, there are approximately 100,000,000 surgeries performed annually in the United States, with millions more worldwide (CDC, National Center for Health Statistics) and these generally have an inherent risk of bleeding, from minor to potentially life threatening. Aside from administration of tranexamic acid for select orthopedic procedures, there are no prophylactic systemic therapies available to administer to help improve hemostasis and minimize surgical bleeding.

Trauma is the third leading cause of death in the United States (CDC, National Center for Health Statistics). A common cause of death following traumatic injury is uncontrolled bleeding (CDC, National Center for Health Statistics). While modern tourniquets are sometimes available to help staunch hemorrhage following extremity trauma, these injuries are still dangerous. Approaches to control non-compressible torso hemorrhage remain even more limited and this is a common cause of death of U.S. soldiers on the battlefield.

Postpartum hemorrhage (PPH) is the leading cause of maternal deaths worldwide. The most common cause is poor contraction of the uterus. Other causes include uterine tears, retained placenta, and inadequate blood clotting. In the United States, approximately 11% of maternal deaths result from PPH, whereas in the developing world approximately 60% of maternal deaths result from PPH. This equates to 100,000 to 140,000 deaths per year. Existing treatments include medications such as oxytocin, misoprostol, and ergotamine, intravenous fluids, blood transfusions, and uterine massage. Surgery to repair cervical or vaginal lacerations or uterine rupture is sometimes necessary as well. Many of these therapeutic options are risky or unavailable in resource-poor areas, resulting in dramatically higher mortality rates.

Hemophilia A is an X-linked recessive disorder associated with spontaneous and prolonged bleeding episodes secondary to deficiencies in clotting factor VIII. More than 20,000 individuals in the United States suffer from this life-long disease. Up to 30% of children with severe hemophilia cannot receive standard factor VIII concentrates due to the development of inhibitor antibodies. Maintaining hemostasis then requires bypassing agents, such as activated prothrombin complex concentrate and recombinant factor VIIa, to help generate clot via alternative pathways. These costly therapies are associated with serious systemic thrombotic side effects, including myocardial ischemia, deep venous thrombosis, and pulmonary embolism. Thus there is a need for new devices, methods, and systems to prevent and treat bleeding problems.

Described herein are devices, methods, and systems that may address the issues identified above.

### SUMMARY OF THE DISCLOSURE

The present invention relates to controlling bleeding in a patient. More specifically, this disclosure is related to apparatuses (devices, systems) and methods for controlling bleeding and bleed time in a patient through coordinated neural stimulation, such as through electrical and/or mechanical stimulation of both the trigeminal and vagal nerves. The apparatus may provide invasive or, preferably, non-invasive stimulation. The stimulation of the vagus nerve may be overlapping, including concurrent, with the trigeminal never, or the trigeminal nerve and vagus nerve may be alternately (with or without overlap) stimulated. The same amount (one or more of: duration, frequency and/or intensity) of stimulation may be applied to both the vagus nerve and the trigeminal nerve, or the amounts (one or more of: duration, frequency and/or intensity) may be different. In some variations Controlling bleeding may include preventing and/or treating bleeding (e.g., surgical bleeding, traumatic bleeding, bleeding related to other medical procedures or conditions, and inherited or acquired bleeding disorders).

For example, described herein methods of reducing bleed time in a subject that include: applying one or more of mechanical or electrical activation to the subject's trigeminal nerve and the subject's vagus nerve; and reducing bleed time of the bleeding by at least 20%.

Also described herein are methods of reducing bleed time in a subject that has been treated with an anticoagulant, the method comprising: applying one or more of mechanical or electrical activation to the subject's trigeminal nerve; and reducing bleed time of the bleeding by at least 20%.

A method of reducing or limiting blood loss in a hemorrhaging subject may include: applying one or more of mechanical or electrical activation to the subject's trigeminal nerve and the subject's vagus nerve; and reducing blood loss from the hemorrhage by at least 10%.

Also described herein are methods of treating a hemophiliac subject, the method comprising: determining when the subject is bleeding; and applying one or more of mechanical or electrical activation to the subject's trigeminal nerve and the subject's vagus nerve to reduce the blood loss and/or bleeding volume.

In any of these methods, the mechanical and/or electrical stimulation may be applied concurrently (e.g., at the same time), or overlapping in time (partially or completely overlapping) and/or within a few second (e.g., within 1 second, 2 seconds, 3 seconds, 4 seconds, 5 seconds, 6 seconds, 10 seconds, etc.). In some variations the intensity of the vagus nerve stimulation may be less than the stimulation of the trigeminal stimulation (e.g., x % or less, where x is 90%, 85%, 80%, 75%, 70%, 65%, 60%, 55%, 50%, 45%, 40%, 35%, 30%, 25%, 20%, 15%, 10%, or 5% of the trigeminal stimulation). In some variations the intensity of the trigeminal nerve stimulation may be less than the stimulation of the vagus stimulation (e.g., x % or less, where x is 90%, 85%, 80%, 75%, 70%, 65%, 60%, 55%, 50%, 45%, 40%, 35%, 30%, 25%, 20%, 15%, 10%, or 5% of the vagus stimulation). Specifically, the intensity may refer to one or more of the applied voltage or current (e.g., when electrical energy is applied), and/or applied force or pressure (when mechanical stimulation is applied). In some variations the intensity may refer to the duration (e.g., the percentage of the total treatment time that the trigeminal stimulation is active vs. the percentage of the total treatment time that the vagus stimulation is active). In some variations intensity may refer to both the applied energy (voltage, force) and the duration of time energy is applied.

In any of these methods, applying may comprise non-invasively applying one or more of mechanical or electrical activation. Although the methods and apparatuses described herein typically refer to non-invasive application of vagus and/or trigeminal stimulation, alternatively, in any of these methods applying may comprise applying from an implant (e.g., implanted neuromodulator that is in communication with the trigeminal and/or vagus nerve).

In any of these methods, the subject may be human or non-human.

Applying may include applying one or more of mechanical or electrical activation to one or more of an ophthalmic, maxillary and/or mandibular branch of the subject's trigeminal nerve in addition to applying one or more of mechanical or electrical activation to the vagus nerve. For example, applying may comprise applying one or more of mechanical or electrical activation to sensory fibers of the patient's trigeminal nerve. In some variation, applying may be limited to applying via the sensory fibers. In some variations, applying comprises applying unilateral activation to the subject's trigeminal nerve. Alternatively, applying may comprise applying bilateral activation to the subject's trigeminal nerve and/or vagus nerve.

Any of these methods may include reducing bleeding time. For example, reducing bleeding time may comprises reducing bleeding time from of one or more of an internal hemorrhage or an external hemorrhage. Bleeding time may be reduced (e.g., the application of electrical and/or mechanical energy may be applied until the bleeding time is reduced) by more than 10%, 15%, 20%, 25%, 30%, 35%, 40%, 45%, 50%, etc.

Similarly, blood loss may be reduced (e.g., the application of electrical and/or mechanical energy which may be applied until the blood loss is reduced) by more than 10%, 15%, 20%, 25%, 30%, 35%, 40%, 45%, 50%, etc.

Applying may comprise applying electrical stimulation to both the vagus nerve and the trigeminal nerve. For example, applying may comprise applying electrical stimulation at between 1-50 Hz and between 0.5-15V having a pulse width of between 0.5 ms and 10 ms to the trigeminal nerve and applying electrical stimulation of between 0.5-100 Hz and between 0.2-15V having a pulse width of between 0.2 ms and 15 ms. Applying may comprise applying for between 1 minute and 45 minutes. Applying may comprise applying electrical stimulation to the trigeminal nerve and mechanical stimulation to the vagus nerve (e.g., in some variations, via the auricular branch).

In any of these methods, applying may comprise applying without triggering a diver's reflex.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 schematically illustrates a system for stimulation both the trigeminal nerve and the vagus nerve. In FIG. 1, the regions (e.g., dermatomes) innervated by the trigeminal nerve are shown one or more of which may be stimulated by the apparatus along with the vagus nerve, shown here schematically applying stimulation to the auricular branch of the vagus nerve.

FIGS. 2A-2H illustrate examples of stimulation patterns for simultaneously stimulating both the vagus nerve and the trigeminal nerve.

FIGS. 3A-3D illustrate examples of systems that may be used to simultaneously stimulate both the vagus nerve and the trigeminal nerve to reduce bleed time.

FIG. 4 is an example of a device for reducing bleed time by simultaneously stimulating both the vagus nerve and the trigeminal nerve.

FIG. 5 schematically illustrates a system for reducing bleed time by simultaneously stimulating both the vagus nerve and the trigeminal nerve.

### DETAILED DESCRIPTION

The present invention relates to controlling (treating and/or preventing) bleeding in a patient. More specifically, this disclosure is related to apparatuses (devices, systems, and methods) for controlling bleeding and controlling (reducing) bleed time in a patient through neural stimulation, such as through electrical and/or mechanical and/or other stimulation of both (e.g., simultaneously) the trigeminal nerve and the vagus nerve. Controlling bleeding may include preventing and/or treating bleeding (e.g., surgical bleeding, traumatic bleeding, bleeding related to childbirth, bleeding related to other medical procedures or conditions, bleeding mediated or increased by anticoagulants, inherited or acquired bleeding disorders such as hemophilia, and so forth).

“Treatment” as used herein includes prophylactic and therapeutic treatment. “Prophylactic treatment” refers to treatment before onset of a condition (e.g., bleeding, an inflammatory condition, etc.) is present, to prevent, inhibit or reduce its occurrence.

As used herein, a patient or subject may be any animal, preferably a mammal, including a human, but can also be a companion animal (e.g., a cat or dog), a farm animal (e.g., a cow, a goat, a horse, a sheep) or a laboratory animal (e.g., a guinea pig, a mouse, a rat), or any other animal.

“Bleed time” or “bleeding time” as used herein refers to the length of time it takes to for bleeding to stop. In general, it is controlled or influenced by how well blood platelets work to form a platelet plug. Bleed time is generally increased by the administration of anticoagulant, such as aspirin, heparin, and warfarin.

As used herein, the terms “reduce” or “reducing” when referring to bleed time in a subject, encompass at least a small but measurable reduction in bleed time over non-treated controls. Reduction may be at least 5%, at least 10%, at least 20%, at least 30%, at least 40%, at least 50%, at least 60%, or more than 60% or anything in between these ranges. For example, a value between these ranges may be chosen so as to use a protocol or apparatus configured to reduce bleeding while minimizing side effects due to applied trigeminal and vagus nerve stimulation.

The nervous system controls nearly every cell and organ in the body through electrical signals carried by nerves. Such electrical connections allow the nervous system to monitor for tissue injury and then to initiate a healing process. Described herein are apparatuses and methods configured for harnessing such electrical connections via targeted electrical nerve stimulation to effectively treat a variety of conditions. Combined vagus and trigeminal nerve stimulation (VNS/TNS) as described herein is a method to reduce bleeding or bleed time following tissue injury or other bleeding event. Combined vagus and trigeminal nerve stimulation (VNS/TNS) as described herein may be non-invasive or minimally invasive. In some examples, VNS/TNS may be a non-invasive or minimally invasive method to activate the vagus nerve and previously described Neural Tourniquet. The combination of vagus nerve stimulation and trigeminal nerve stimulation may reduce the amount of one or both vagus and trigeminal nerve stimulation necessary for robust reduction of bleed time.

“Combined” vagus and trigeminal nerve stimulation (“VNS/TNS”) may refer to the simultaneous (e.g., at the same time), overlapping or near-overlapping (e.g., within about 10seconds or less, e.g., within 9 sec or less, 8 sec or less, 7 sec or less, 5 sec or less, 2 sec or less, 1 second or less, 0.5 seconds or less, etc.) vagus and trigeminal stimulation.

“Non-invasive stimulation” typically means stimulation that does not require a surgery, exposure of the nerve fiber or direct contact with the nerve fiber. As used herein, “non-invasive stimulation” also does not include administration of pharmacological agents. For example, non-invasive trigeminal nerve stimulation can be achieved, for example, by mechanical (e.g., vibration) or electrical (e.g., electromagnetic radiation) means applied externally to the subject. Similarly non-invasive vagus nerve stimulation may be achieved, for example, by electrical or mechanical (e.g., vibration) stimulation applied externally (e.g., to the auricular region of the ear, over the auricular branch of the vagus nerve.

Although in some examples, a non-invasive or minimally invasive approach as described herein may be used in conjunction with a pharmacological approach (e.g., for an additive or a synergistic benefit), in general an approach described herein may be more efficacious, safer, and less costly than traditional pharmacological therapies. Advantages of this method over pharmacological approaches may include higher specificity, fewer side effects, lower costs, and improved compliance. Advantages over implantable pulse generators for chronic nerve stimulation applications may include avoidance of surgery and associated complications, both for the initial procedure and subsequent procedures for battery changes, and lower costs.

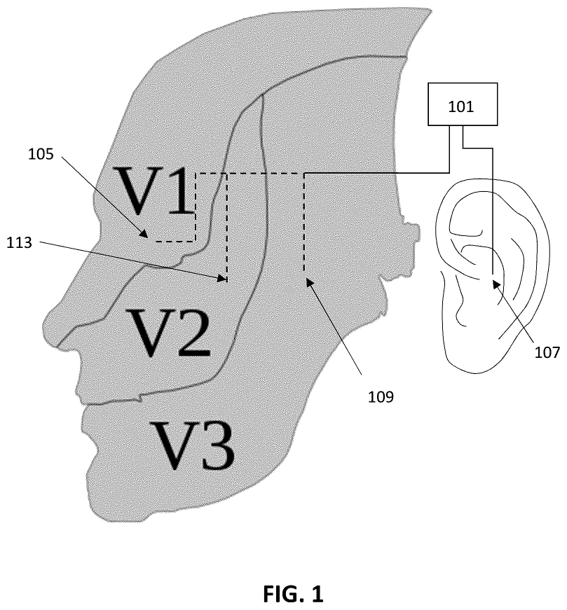

The trigeminal nerve (cranial nerve V) is the largest of the cranial nerves, and has three different branches or nerve distributions (V1, V2, V3; also referred to as the ophthalmic nerve, maxillary nerve and mandibular nerve, respectively) that converge on the trigeminal ganglion. The trigeminal nerve is paired and present on both sides of the body. The trigeminal nerve relays sensory (and motor) information from the head and face. Trigeminal nerve stimulation (TNS) is thought to activate multiple structures in the brain and brainstem, such as the locus coeruleus (LC) and nucleus tractus solitarius (NTS). FIG. 1 shows a schematic of the different skin regions corresponding to the different branches of the trigeminal nerve. The vagus nerve (cranial nerve X) is the longest of the cranial nerves, extending from the brainstem down into the peritoneal cavity. The vagus nerve is the main parasympathetic output of the autonomic nervous system, and interfaces with nearly every organ of the thorax and abdomen, including the heart, lungs, liver, and spleen. Vagus nerve stimulation (VNS) is clinically approved for the treatment of medically refractory epilepsy and depression. Activation of the LC and NTS appears important to the antiepileptic effects of VNS. To date, more than 100,000 patients have received VNS. Technological advances may allow for nerve stimulation without surgical implantation of a pulse generator. For example, transcutaneous auricular stimulation demonstrates anticonvulsive effects similar to invasive VNS.

Direct electrical stimulation of the cervical vagus nerve significantly shortens the duration of bleeding and decreases total blood loss during tissue trauma in swine. Rotational thromboelastography (RoTEG) revealed that VNS significantly shortens the reaction (r) time of blood to initiate clot formation. Moreover, VNS significantly increases thrombin generation at the injury site, whereas systemic thrombin production remains unchanged. Taken together, VNS improves hemostasis by accelerating clot formation specifically at the site of tissue injury.

As described herein VNS/TNS (combined vagus and trigeminal stimulation) may include activating the trigeminal nerve (e.g., by electrical or mechanical or other stimulation) and activating the vagal nerve directly. For example, the vagus nerve may be activated directly in combination with trigeminal nerve activation. Thus a step of controlling bleeding or activating the trigeminal nerve may include a step of directly activating the vagal nerve. Activating the trigeminal nerve may include activating the cholinergic anti-inflammatory pathway and/or any other steps to control bleeding or bleed time in a subject as described in U.S. Pat. No. 8,729,129, while concurrently directly stimulating the vagus nerve. The vagal nerve may be activated either directly or indirectly. In some particular examples, activating the vagus nerve-mediated reduced bleed-time safely and efficaciously may be through stimulation of the trigeminal nerve and the vagus nerve, utilizing precise and specific electrical stimulation parameters. Trigeminal nerve and vagus nerve stimulation may include improving hemostasis via accelerated clot formation such as at the site of tissue injury. This may lead to less blood loss and a shorter duration of bleeding following tissue trauma and hemorrhage.

FIG. 1 shows one example of a schematic for a system configured for the combined stimulation of the vagus and trigeminal nerves. In FIG. 1, the system may include a controller 101 that may include control logic and/or circuitry for driving combined stimulation of the vagus nerve using a vagus nerve stimulation output 107 and a trigeminal nerve stimulation output. In FIG. 1, the trigeminal nerve is shown with three alternatively stimulation outputs 105, 109, 113. One or more branch of the trigeminal nerve may be stimulated by the system; for example, in FIG. 1, the V1 branch of the trigeminal nerve may be mechanically or electrically stimulated by a stimulation output 105 of the system. The V2 branch of the trigeminal nerve may be mechanically or electrically stimulated by a stimulation output 113 of the system. The V3 branch of the trigeminal nerve may be mechanically or electrically stimulated by a stimulation output 109 of the system. For example, one or more electrodes configured to contact the patient's skin over the V1, V2 or V3 branch of the trigeminal nerve may be included. Electrical stimulation may be applied (e.g., pulsed electrical stimulation of between 1-4 kHz at a current of between 0.1 mA to 100 mA). Similarly, the system may include a vagus nerve stimulator 107 that may be connected to the controller 101 to drive stimulation of the vagus nerve. The vagus nerve stimulation may be mechanical stimulator (e.g., configured to apply mechanical force/pressure to the vagus nerve from outside of the body, e.g., by applying against the patient's ear) or an electrical stimulator. For example, the electrical vagus nerve stimulators may apply electrical stimulation from one or more electrodes on the surface of the patient's skin (e.g., the auricular region of the ear). In some variations the electrode may be one or more tissue penetrating electrodes (e.g., needles) inserted into the skin.

In either the vagus or trigeminal stimulators, the apparatus may include a patch (e.g., patch electrode) for contacting part of the body (e.g., head, ear, face, etc.) of a subject and delivering a pulse and a stimulator for providing an electrical stimulus to be delivered through the patch.

Any appropriate electrical or mechanical stimulation may be applied. For example, when applying electrical stimulation to the trigeminal nerve (e.g., through the face), a voltage stimuli (e.g., between 0.2 V to 5 V, at between 0.1-50 Hz, between 0.1 ms and 5 ms pulse width, monophasic and/or biphasic) may be applied for x min (e.g., where x is 2 min, 5 min, 10 minutes, 20 minutes, or 30 minutes, etc.) duration. Vagus nerve stimulation may be applied at approximately or exactly the same time. For example, one complete operational cycle (“dose”) may include a 0.2 V-5 V monophasic pulses (e.g., sinusoidal, rectangular, etc. pulses) for a burst duration that is continuous or repeating, with pulses having a duration of between 0.1 ms and 10 ms (e.g., 2 milliseconds). This cycle may be repeated at a repetition rate of between about 0.1 Hz and 1000Hz (e.g., 30 Hz) for a treatment duration of between 1 min and 40 min (e.g., 10 minutes, 20 minutes, or 30 minutes, etc.). Concurrently stimulation of the vagus nerve may be applied, e.g., through the ear. For example, stimulation of between about 0.1-10V, 0.1-10 mA, pulsed, e.g., rectangular pulses, for a burst duration that is continuous or repeating, with pulses having a duration of between 0.1 ms and 10 ms (e.g., 2 milliseconds). This cycle may be repeated at a repetition rate of between about 0.1 Hz and 1000Hz (e.g., 30 Hz) for a treatment duration of between 1 min and 40 min (e.g., 10 minutes, 20 minutes, or 30 minutes, etc.).

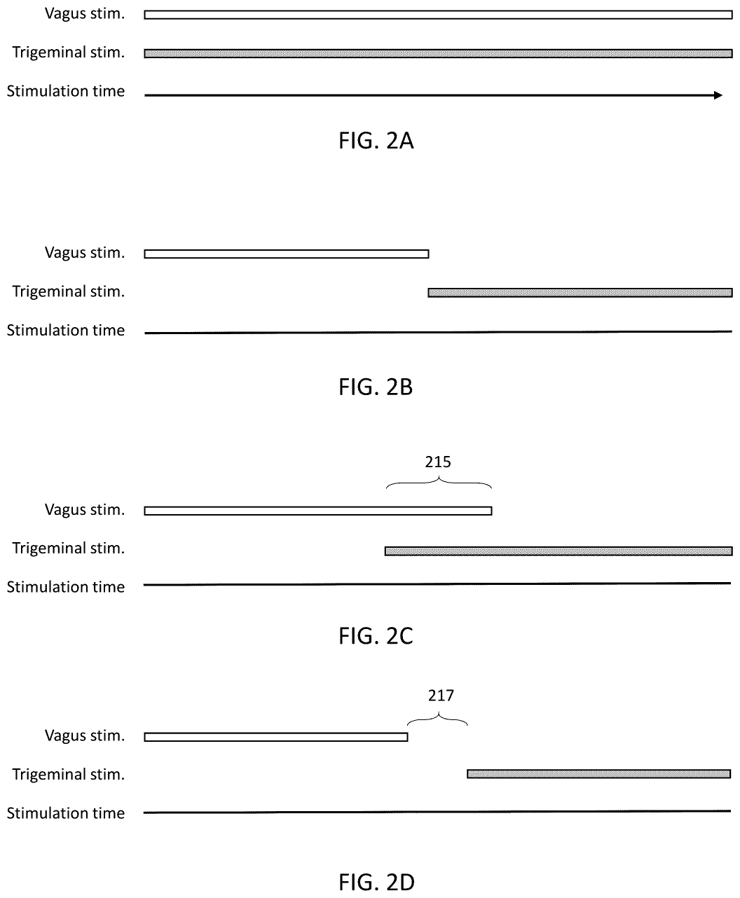

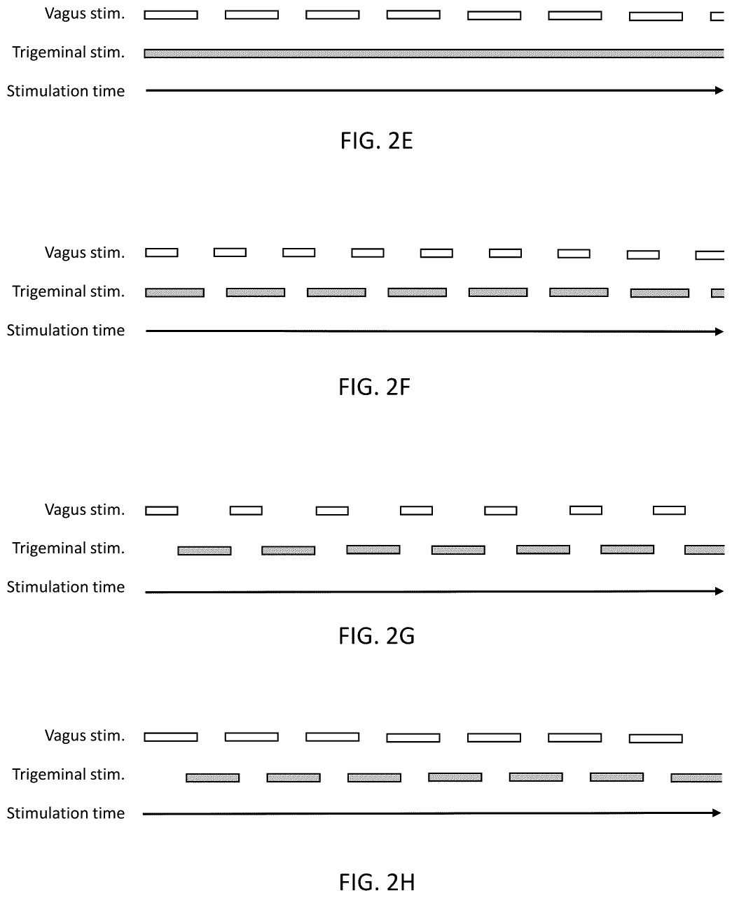

The pattern of concurrent stimulation for the vagus and the trigeminal may be arranged in a variety of different ways. For example, FIGS. 2A-2H illustrate variations of the combined vagus and trigeminal stimulation. In FIG. 2A, the vagus and trigeminal nerve are stimulated at the same time (e.g., same start and stop). This simulation may be identical in frequency (e.g., puling, etc.), and/or intensity (e.g., amplitude, burst duration, etc.). For example, in variations in which both vagus and trigeminal are stimulated electrically by pulsed electrical stimulation, the stimulation may occur at the same time, as shown (e.g., having the same start/stop). Alternatively, in some variations combined vagus nerve and trigeminal nerve stimulation to reduce bleed time may include first stimulating the trigeminal nerve followed by stimulation of the vagus nerve, or by first stimulating the vagus nerve, followed by stimulation of the trigeminal nerve, as shown in FIG. 2A. This alternating stimulation may be repeated for the entire dose duration. In FIG. 2A there is no significant gap between the vagus stimulation and the trigeminal stimulation; in some variations, as shown in FIG. 2D, the combined vagus/trigeminal stimulation includes a gap 217 between the vagus and trigeminal nerve stimulation. As mentioned, this gap may be less than a few second (e.g., 10 seconds or less, 9 seconds or less, 8 seconds or less, 7 seconds or less, 6 seconds or less, 5 seconds or less, 4 seconds or less, 3 seconds or less, 2 seconds or less, 1 seconds or less, 0.5 seconds or less, etc.). The vagus/trigeminal nerve stimulation may therefore alternate and may be repeated for the entire dose duration.

Alternatively, in some variations, as shown in FIG. 2C, the combined vagus and trigeminal stimulation may include overlapping 215 stimulation of the trigeminal and vagus nerve, as shown. In any of these variations, vagus nerve stimulation may begin before trigeminal nerve stimulation (as shown) or in some variations, trigeminal nerve stimulation may begin before vagus nerve stimulation.

In some variations, either vagus nerve stimulation or trigeminal nerve stimulation may be intermittent and overlap with constant stimulation of the trigeminal (when vagus is intermittent) or vagus (when trigeminal stimulation is intermittent). In FIG. 2E the trigeminal nerve is stimulated continuously (although this may include pulsed or burst of pulses) while the vagus nerve stimulation is intermittent (e.g., turned “on” and “off” with an intermittence frequency) during the dose duration.

In some variations, as shown in FIG. 2F-2H, combined vagus and trigeminal nerve stimulation to reduce bleeding (e.g., reduce bleed time) may include both vagus nerve stimulation and trigeminal nerve stimulation being pulsed on/off at the same or different frequencies. In FIG. 2F, the vagus nerve stimulation may be performed at an on/off frequency (intermittence frequency) that is different than the trigeminal nerve stimulation frequency; in this example the vagus nerve stimulation has a duty cycle of approximately 50%, while the trigeminal nerve stimulation has a duty cycle of >50% (e.g., >60%, approximately 75%). The vagus nerve stimulation may partially overlap with the trigeminal nerve stimulation during the dose duration, or may not.

In FIG. 2G the combined vagus and trigeminal stimulation to reduce bleeding may include alternating periods of vagus and trigeminal stimulation in which either the vagus nerve stimulation is on for longer than the trigeminal nerve stimulation or the trigeminal nerve stimulation is on for longer than the vagus nerve stimulation (as shown in FIG. 2G). In FIG. 2H, both trigeminal and vagus nerve stimulation are on for the same duration, and the trigeminal and vagus nerve stimulation ‘on’ times overlap.

In general, the non-invasive stimulation described herein may be non-invasive electrical stimulation applied at a predetermined range of intensities and frequencies. However, other types of non-invasive stimulation may also be used (e.g. non-invasive mechanical stimulation) and can minimally invasive, subcutaneous stimulation. Non-invasive stimulation may be performed by one or more electrodes or actuators that do not contact the nerve. Electrical stimulation may be in the range of 10 mV to 5 V at a frequency of 0.1 Hz to 100 Hz, with a duration of stimulus between from 1 ms to 10 min.

Mechanical stimulation may be oscillatory, repeated, pulsatile, or the like. In some variations the non-invasive stimulation may the repeated application of a mechanical force against the subject's skin at a predetermined frequency for a predetermined period of time. For example, the non-invasive mechanical stimulation may be a mechanical stimulation with a spectral range from 50 to 500 Hz, at an amplitude that ranges between 0.0001-5 mm displacement. The temporal characteristics of the mechanical stimulation may be specific to the targeted disease. In some variations the frequency of stimulation is varying or non-constant. The frequency may be varied between 50 and 500 Hz. In some variations the frequency is constant. In general the frequency refers to the frequency of the pulsatile stimulation within an “on period” of stimulation. Multiple stimulation periods may be separated by an “off period” extending for hours or even days, as mentioned above.

The force with which the mechanical stimulation is applied may also be constant, or it may be variably. Varying the force and/or frequency may be beneficial to ensure that the mechanical stimulation is effective during the entire period of stimulation, particularly if the effect of non-invasive stimulation operates at least in part through mechanoreceptors such as the rapidly acclimating Pacinian corpuscles.

In performing any of the therapies described herein, the non-invasive stimulation may be scheduled or timed in a specific manner. For example, a period of stimulation (“on stimulation”) may be followed by a period during which stimulation is not applied (“off period”). The off period may be much longer than the on period. For example, the off period may be greater than an hour, greater than two hours, greater than four hours, greater than 8 hours, greater than 12 hours, greater than 24 hours, or greater than 2 days. The on period is the duration of a stimulation (which may include a frequency component), and may be less than 10 minutes, less than 5 minutes, less than 2 minutes, less than 1 minute, etc. The ratio of the on period and the off period may partially determine the duty cycle of stimulation.

In some examples, either one (e.g., left or right) of the two paired trigeminal nerves may be activated (e.g., unilateral activation). In some examples, the paired trigeminal nerves may be both be activated in a subject (e.g., bilateral activation). In some examples, part or all of the trigeminal nerve may be activated. For example, any one, two or three of the three different branches or nerve distributions (V1, V2, V3; also referred to as the ophthalmic nerve, maxillary nerve and mandibular nerve, respectively) may be activated. In some examples, sensory fibers of the trigeminal nerve are stimulated. Additionally, the trigeminal ganglion may also or instead be stimulated. Additionally or instead, associated neurons that are connected to the trigeminal nerve may be stimulated.

Stimulation may be performed using one or more patches configured to cover part of the body each containing one or more electrodes (an array of 2, 3, 4, 5, 10, or more electrodes) configured to cover part of the body (e.g. cheek, forehead, head, neck, nose, scalp, etc.) in a position sufficient to provide stimulation one or more parts of a trigeminal nerve. Stimulation may be performed using one or more electrodes configured to be placed under the skin, such as in a muscle and 1, 2, 3, 4, 5, 10, or more electrodes) may be placed in a muscle.

Also described herein are apparatuses (devices, systems, and methods) for activating the trigeminal nerve and the vagal nerve. In some embodiments, both the trigeminal nerve and the vagal nerve may be directly activated (e.g., by electrical, mechanical or other stimulation such as magnetic, thermal, etc.).

Further, in some variations, the trigeminal stimulation described herein may not activate the dive reflex. The dive reflex in general can activated, for example, by submerging the body in cold water (and holding the breath) wherein the body overrides basic homeostatic functions. The dive reflex is a physiological adaptation that regulates respiration, heart rate, and arterial blood pressure in a particular way. Although all mammals control breathing, heart rate, and arterial blood pressure during their lives, these controls are strongly altered during diving and activation of the dive reflex. In general, trigeminal stimulation parameters may be chosen so as to not activate the dive reflex (e.g., trigeminal stimulation without inducing a dive reflex). Failure to induce a dive reflex may be failure to invoke a percentage change in heart rate and/or respiration and/or arterial blood pressure by more than a predetermined amount. For example, failure to induce a dive reflex may be failure to reduce one or more of heart rate and/or respiration and/or arterial blood pressure by greater than about 2%, 5%, 7%, 10%, 15%, 20%, 25%, 30%, 40%, etc.

The apparatuses and methods described herein may be suitable for therapeutically or prophylactically treating subjects suffering from or at risk from suffering from unwanted bleeding from any cause such as: bleeding disorders including but not limited to afibrinogenemia, Factor II deficiency, Factor VII deficiency, fibrin stabilizing factor deficiency, Hageman Factor deficiency, hemophilia A, hemophilia B, hereditary platelet function disorders (e.g., Alport syndrome, Bernard-Soulier Syndrome, Glanzmann thrombasthenia, gray platelet syndrome, May-Hegglin anomaly, Scott syndrome, and Wiskott-Aldrich syndrome), parahemophilia, Stuart Power Factor deficiency, von Willebrand disease, thrombophilia, or acquired platelet disorders (such as those caused by common drugs: antibiotics, and anesthetics, blood thinners, and those caused by medical conditions such as: chronic kidney disease, heart bypass surgery, and leukemia), childbirth, injury, menstruation, and surgery. An unwanted bleeding treated using any of the apparatuses or methods described herein may include an internal hemorrhage or an external hemorrhage. An internal hemorrhage includes a hemorrhage in which blood is lost from the vascular system inside the body, such as into a body cavity or space. An external hemorrhage includes blood loss outside the body.

### EXAMPLES

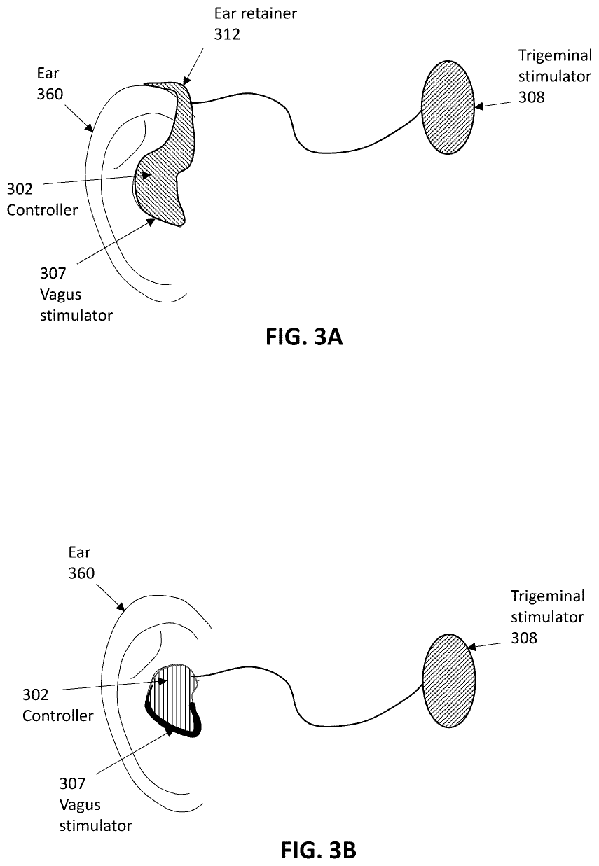

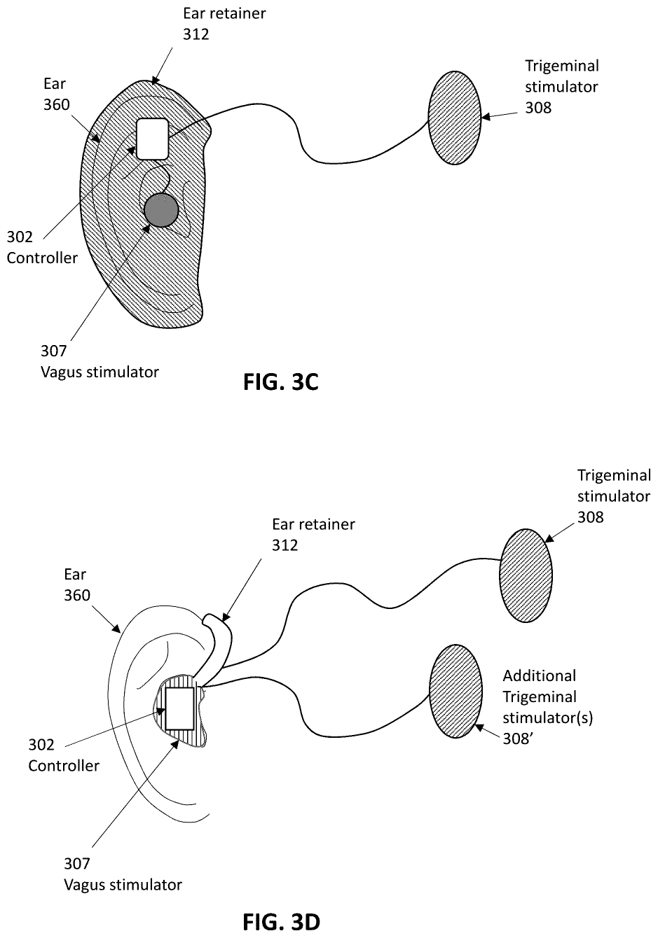

FIG. 3A illustrates one example of a combined trigeminal and vagus nerve stimulator for treating bleeding (e.g., for reducing bleed time) as described. In FIG. 3A, the apparatus includes a housing that is configured or adapted to fit over, behind and at least partially into the patient's auricle region of the ear. The housing may include an ear retainer 312 for holding the device in/on the ear 360, and may at least partially enclose a controller (e.g., control circuitry, a battery, power control circuitry, waveform generator, a trigeminal stimulation drive and vagus stimulation drive). The apparatus also includes a vagus stimulator 307 that is coupled to the housing in this example, to be applied against the patient's ear. A connector (e.g., cable, wire, etc.) connects a trigeminal stimulator 308 that may be worn on the patient's face (e.g., in the V1, V2 and/or V3 region, as shown in FIG. 1). The controller may be connected (via a wire or wireless connection) to a user interface that may control starting/stopping of the dose, or in some variations the housing may include a control (e.g., button, dial, etc.). The dose may be preprogrammed into the controller and/or it may be adjusted.

FIG. 3B shows another example of a combined trigeminal and vagus nerve stimulator for treating bleeding (e.g., for reducing bleed time) as described. In FIG. 3B, the apparatus includes a housing that is configured or adapted to fit at least partially into the patient's ear, as shown. The housing may be held in the ear 360, and may include a foam or other expandable material to help secure it in place. Alternatively a separate retainer may be used to hold it in/on the ear (not shown). The housing may at least partially enclose a controller (e.g., control circuitry, a battery, power control circuitry, waveform generator, a trigeminal stimulation drive and vagus stimulation drive). The apparatus may also include a vagus stimulator 307 that is coupled to the housing in this example, to be applied against the patient's ear. A connector (e.g., cable, wire, etc.) connects a trigeminal stimulator 308 that may be worn on the patient's face (e.g., in the V1, V2 and/or V3 region, as shown in FIG. 1). The controller may be connected (via a wire or wireless connection) to a user interface that may control starting/stopping of the dose, or in some variations the housing may include a control (e.g., button, dial, etc.). The dose may be preprogrammed into the controller and/or it may be adjusted.

FIG. 3C is another example of a combined trigeminal and vagus nerve stimulator for treating bleeding (e.g., for reducing bleed time) as described. In FIG. 3C, the apparatus include an ear retainer 312 that is configured or adapted to fit at least partially over the patient's ear, as shown. The retainer holds the device over the patient's ear 360, so that the vagus stimulator 307 is in contact with the region of the ear over the vagus nerve. The retainer also holds the controller 302 and may be formed of a material (e.g., mesh, etc.) that fits over the ear to help secure it in place. The controller (e.g., control circuitry, a battery, power control circuitry, waveform generator, a trigeminal stimulation drive and vagus stimulation drive) may be held by the retainer; the vagus nerve stimulator may include a biocompatible adhesive (e.g., hydrogel, etc.) for making electrical contact with the ear. A connector (e.g., cable, wire, etc.) connects the controller with a trigeminal stimulator 308 that may be worn on the patient's face (e.g., in the V1, V2 and/or V3 region, as shown in FIG. 1). The controller may be connected (via a wire or wireless connection) to a user interface that may control starting/stopping of the dose, or in some variations the housing may include a control (e.g., button, dial, etc.). The dose may be preprogrammed into the controller and/or it may be adjusted.

FIG. 3D is another example of a combined trigeminal and vagus nerve stimulator for treating bleeding (e.g., for reducing bleed time) as described. In FIG. 3D, the apparatus is configured or adapted to fit at least partially into the patient's ear, as shown. The apparatus may be held in the ear 360 by an ear retainer 312 to secure it in place. The retainer may fit over the back of the ear and/or partially under the ear to hold the apparatus in/on the ear. In FIG. 3D, the controller (e.g., control circuitry, a battery, power control circuitry, waveform generator, a trigeminal stimulation drive and vagus stimulation drive) is shown on the front; in some variations the controller may be on the back of the apparatus (e.g., held behind the ear). The apparatus may also include a vagus stimulator 307 that is configured to contact the ear. A connector (e.g., cable, wire, etc.) connects the controller to a first trigeminal stimulator 308 that may be worn on the patient's face (e.g., in the V1, V2 and/or V3 region, as shown in FIG. 1). One or more additional trigeminal stimulators 308′ may be connected as well (e.g., in parallel or in series with the first trigeminal stimulator). Thus, multiple sites may be used for trigeminal stimulation. The controller may be connected (via a wire or wireless connection) to a user interface that may control starting/stopping of the dose, or in some variations the housing may include a control (e.g., button, dial, etc.). The dose may be preprogrammed into the controller and/or it may be adjusted.

In any of these apparatuses, the vagus stimulator (vagus nerve stimulator) may be an electrical or a mechanical stimulator. In variations in which the apparatus is an electrical stimulator, the vagus stimulator may include one or more electrodes that may be coupled to the patient's skin and/or may penetrate into the skin (e.g., as shallow needle electrodes). The electrodes may apply electrical energy to modulate the vagus nerve, as descried herein. Mechanical stimulators may apply mechanical energy as described above. Similarly, any of these apparatuses may include one or more trigeminal stimulators that may be configured to apply electrical stimulation (e.g., including one or more electrodes, which may include a hydrogel for making skin contact). The trigeminal stimulators may alternatively be mechanical stimulators.

In any of the methods and apparatuses described herein, VNS/TNS can modulate both the patient's sympathetic nervous system (SNS) and parasympathetic nervous system (PNS) activities to reduce bleed time.

As mentioned above, any of these methods and apparatuses may be configured to non-invasively applying neuromodulation of the trigeminal nerve and vagus nerve. Alternatively or additionally, invasive (e.g., using a needle electrode, implant, etc.) may be used for either VNS, TNS or both VNS and TNS.

For example, non-invasive trigeminal stimulation may be applied via one or more skin surface electrodes that apply trigeminal stimulation to one or more of the subject's forehead, cheek(s), nose, tongue, or other facial skin. In some embodiments, applying the non-invasive neurostimulation to the subject's trigeminal nerve includes targeting at least one of the ophthalmic nerve, maxillary nerve, or mandibular nerve. Alternatively, in some variations, applying non-invasive neurostimulation to the subject's trigeminal nerve includes avoiding targeting at least one of the ophthalmic nerve, maxillary nerve, or mandibular nerve.

Any appropriate frequency and/or amplitude and/or duration may be used. In some embodiments, applying the non-invasive neurostimulation to the subject's trigeminal nerve comprises non-invasive neurostimulation has a frequency of 1-300 (e.g., between 10-60 Hz, etc.). In some embodiments, the non-invasive neurostimulation has an intensity of 2 mV-20 V (e.g., between 0.5 V and 15V, between 1 V and 12 V, etc.). In some embodiments, the non-invasive neurostimulation has a duty cycle of between about 20% to 70% (e.g., 1 second “on” and 1-2 seconds “off”). In some embodiments, the non-invasive neurostimulation includes a pulse width of between about 0.1 ms to 10 ms (e.g., between about.1 ms to 5 ms, between about 0.25 to 5 ms, etc.). In some embodiments, at least one of a stimulation voltage or a current is increased gradually (e.g., steps of 0.1 V). In some embodiments, the closed-loop trigeminal and/or vagus nerve stimulation is conducted based on a heart rate of the patient (e.g., subject). In some embodiments, the closed-loop trigeminal nerve stimulation is conducted based on a heart rate variability (HRV) of the patient. In some embodiments, certain parameters of the stimulation are modulated to maintain values of the parameters within a target range (e.g., preventing a hear rate or blood pressure effect, etc.).

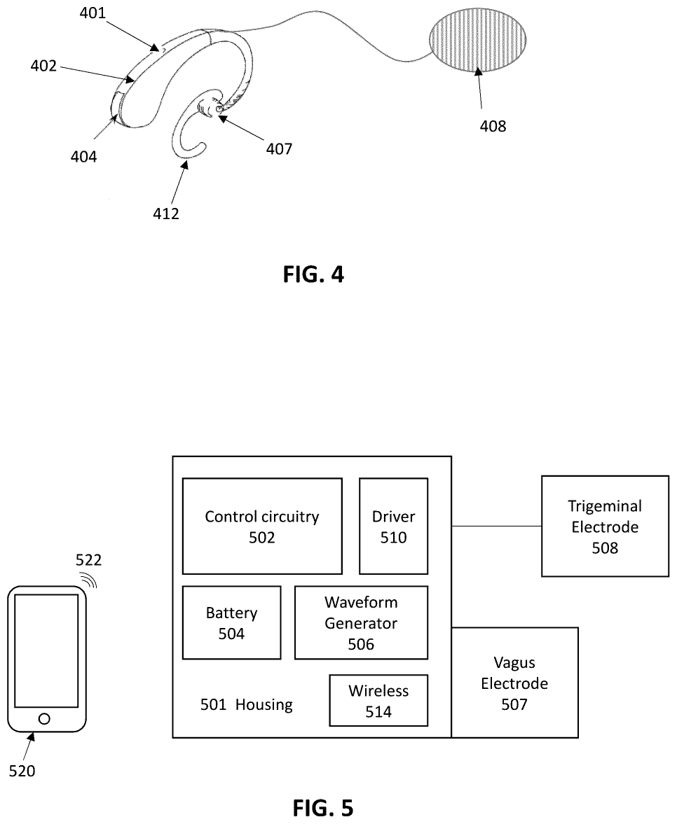

FIG. 4 is another example of a combined vagus and trigeminal nerve stimulator for reducing bleeding (e.g., reducing bleed time). In FIG. 4, the apparatus includes a housing 401 enclosing a controller 402 (e.g., control circuitry) and a battery 404. The housing is configured to fit behind a patient's ear (not shown), and insert a vagus stimulator 407 into the ear so that it is in contact with the region above the auricular branch of the vagus nerve. In FIG. 4 the apparatus also includes an additional retainer 412 to help anchor the vagus stimulator. A trigeminal stimulator 408 is connected to the controller as well (the connection shown in a wire). The trigeminal stimulator may be an electrode pad that is in electrical communication with the controller (e.g., driver, waveform generator, etc.); similarly the vagus stimulator may include an electrode (or electrode pad) that is in electrical communication with the controller.

FIG. 5 schematically illustrates some of the components of an apparatus for combined vagus and trigeminal stimulation to reduce bleeding as described above in FIGS. 3A-4. In the schematic of FIG. 5, the controller 502 may be separate from or integrated with one or more drivers 510 and waveform generators 506 that may generate and provide power to the trigeminal stimulator (e.g., shown here as a trigeminal electrode 508) and vagus stimulator (shown as a vagus electrode 507). The controller may also be connected to or include wireless communication circuitry 514 for wirelessly communicating 522 with one or more external devices 520 (shown in this example as a smartphone, though any external processor may be used). In FIG. 5, the controller (including control circuitry) may be housed within a housing 501. In some variations this housing may be configured or adapted to fit into, on and/or over a patient's ear (generically referred to as on the patient's ear).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. (canceled)

2. A method for treating or preventing bleeding in a subject, the method comprising:
positioning a first electrical or mechanical neuromodulation stimulator in piercing or non-piercing contact with a facial region of skin of the subject in a first location targeting a trigeminal nerve, wherein the first electrical or mechanical neuromodulation stimulator is operably connected to a trigeminal stimulation drive of a controller;
positioning a second electrical or mechanical neuromodulation stimulator in piercing or non-piercing contact with an auricular region and/or head region of skin of the subject in a second location targeting a vagus nerve, wherein the second electrical or mechanical neuromodulation stimulator is operably connected to a vagal stimulation drive of the controller; and
activating the controller to deliver a neurostimulation treatment configured for treating and/or preventing bleeding, wherein delivering the neurostimulation treatment comprises applying
i) via the trigeminal stimulation drive, first neurostimulation activations at a first frequency or range of frequencies, and
ii) via the vagal stimulation drive, second neurostimulation activations at a second frequency or range of frequencies,
wherein the first neurostimulation activations and the second neurostimulation activations are configured to modulate activities of a sympathetic nervous system (SNS) and a parasympathetic nervous system (PNS) of the subject, thereby shortening a reaction time to form a clot.

3. The method of claim 2, wherein targeting the trigeminal nerve comprises targeting at least one of an ophthalmic nerve branch, a maxillary nerve branch, or a mandibular nerve branch.

4. The method of claim 2, wherein:
the first neurostimulation activations are delivered via the first electrical neuromodulation stimulator; and
the first frequency or range of frequencies is within a range from 31 Hz to 150 Hz.

5. The method of claim 2, wherein:
the second neurostimulation activations are delivered via the second electrical neuromodulation stimulator; and
the second frequency or range of frequencies is within a range from 1 Hz to 30 Hz.

6. The method of claim 2, wherein:
the first neurostimulation activations are delivered via the first mechanical neuromodulation stimulator; and
the first frequency or range of frequencies is within a range from 75 Hz to 500 Hz.

7. The method of claim 2, wherein:
the second neurostimulation activations are delivered via the second mechanical neuromodulation stimulator; and
the second frequency or range of frequencies is within a range from 20 Hz to 500 Hz.

8. The method of claim 2, wherein the neurostimulation treatment is a prophylactic treatment.

9. The method of claim 2, wherein the first neurostimulation activations and the second neurostimulation activations are configured to increase thrombin generation at an injury site while maintaining a systemic thrombin level in the subject.

10. The method of claim 9, wherein the injury site is an internal injury site.

11. The method of claim 2, wherein the first neurostimulation activations and the second neurostimulation activations are configured to reduce bleed time by at least 10%.

12. The method of claim 2, wherein the controller is activated to deliver the neurostimulation treatment via a user interface of the controller.

13. The method of claim 2, further comprising adjusting a dose of the neurostimulation treatment responsive to receiving input via a user interface of the controller.

14. A system for treating or preventing bleeding in a subject, the system comprising:
a controller;
a trigeminal stimulation drive;
a vagal stimulation drive;
a first electrical or mechanical neuromodulation stimulator configured to be positioned in piercing or non-piercing contact with a facial region of skin of the subject in a first location targeting a trigeminal nerve, wherein the first electrical or mechanical neuromodulation stimulator is operably connected to the trigeminal stimulation drive; and
a second electrical or mechanical neuromodulation stimulator configured to be positioned in piercing or non-piercing contact with an auricular region and/or head region of skin of the subject in a second location targeting a vagus nerve, wherein the second electrical or mechanical neuromodulation stimulator is operably connected to the vagal stimulation drive;
wherein the controller comprises control circuitry configured to deliver a neurostimulation treatment for treating and/or preventing bleeding, wherein delivering the neurostimulation treatment comprises applying
i) via the trigeminal stimulation drive, first neurostimulation activations at a first frequency or range of frequencies, and
ii) via the vagal stimulation drive, second neurostimulation activations at a second frequency or range of frequencies,
wherein the first neurostimulation activations and the second neurostimulation activations are configured to modulate activities of a sympathetic nervous system (SNS) and a parasympathetic nervous system (PNS) of the subject, thereby shortening a reaction time to form a clot.

15. The system of claim 14, wherein the controller further comprises a waveform generator.

16. The system of claim 14, further comprising a housing, wherein:
the first electrical or mechanical neuromodulation stimulator and the second electrical or mechanical neuromodulation stimulator are arranged on an exterior of the housing; and
the trigeminal stimulation drive and the vagal stimulation drive are enclosed within the housing.

17. The system of claim 16, wherein the housing is configured to be positioned on facial skin of the subject.

18. The system of claim 16, wherein the housing is configured to be positioned on and/or proximate to an ear of the subject.

19. The system of claim 14, wherein the first electrical or mechanical neuromodulation stimulator and the second electrical or mechanical neuromodulation stimulator are wirelessly connected to the controller.

20. The system of claim 14, wherein delivering the neurostimulation treatment comprises alternating applying the first neurostimulation activations with applying the second neurostimulation activations.

21. The system of claim 14, wherein the neurostimulation treatment is configured to treat menstruation.

22. The system of claim 14, wherein the first mechanical neuromodulation stimulator and the second mechanical neuromodulation stimulator are configured to deliver vibrational stimulation.
